(19) United States
(12) Patent Application Publication
(10) Pub. No.: US 2021/0338109 A1
(43) Pub. Date: Nov. 4, 2021

(54) FATIGUE DETERMINATION DEVICE AND FATIGUE DETERMINATION METHOD

(71) Applicant: MITSUBISHI ELECTRIC CORPORATION, Tokyo (JP)

(72) Inventor: Hirofumi NISHIKAWA, Tokyo (JP)

(73) Assignee: MITSUBISHI ELECTRIC CORPORATION, Tokyo (JP)

(21) Appl. No.: 17/372,840

(22) Filed: Jul. 12, 2021

Related U.S. Application Data
(63) Continuation of application No. PCT/JP2019/005804, filed on Feb. 18, 2019.

(51) Int. Cl. A61B 5/11

(52) U.S. Cl. CPC A61B 5/1107; A61B 5/1128; A61B 5/112

(57) ABSTRACT

A skeleton extraction unit (120) extracts skeleton information expressing in a time series a motion of a skeleton of a person from two-dimensional video data (161) obtained by capturing a walking movement of the person. A walk analysis unit (130) calculates walk analysis data (31) including arm swing information expressing an arm swing state of the person in walking and gait information expressing a gait state of the person in walking, using the video data (161). A determination unit (150) compares a determination threshold value (164) for determining a fatigue degree of the person with the walk analysis data (31) of the person, and determines the fatigue degree of the person using a comparison result. The determination threshold value (164) includes a threshold value of the arm swing information and a threshold value of the gait information.

### CROSS REFERENCE TO RELATED APPLICATIONS

This application is a Continuation of PCT International Application No. PCT/JP2019/005804 filed on Feb. 18, 2019, which is hereby expressly incorporated by reference into the present application.

### TECHNICAL FIELD

The present invention relates to a fatigue determination device, a fatigue determination method, and a fatigue determination program.

### BACKGROUND ART

As a conventional technique, there is a fatigue determination device which detects a user's physical condition or fatigue.

With a physical condition detection device of Patent Literature 1, a walk analysis result of a user is recorded. In the physical condition detection device of Patent Literature 1, a detection-target user is photographed by a depth camera capable of measuring a depth of each pixel, walk analysis of the user is performed on the basis of the depth of each pixel, and an analysis result is compared with the recorded walk analysis result. Then, the physical condition detection device of Patent Literature 1 identifies a physical condition of the user by determining an occurrence of a change that satisfies a condition.

Further, in Patent Literature 2, a method that does not use a depth camera is disclosed, which attaches a marker to a person, detects the marker by a tracker such as an ordinary camera, and processes the detected marker, thereby digitally recording a motion of the person. Alternatively, a method is disclosed which measures a distance from a sensor to a person using an infrared sensor, and detects a size of the person and various motions such as a motion of a skeleton of the person.

### CITATION LIST

### Patent Literature

Patent Literature 1: JP 2017-205134 A

Patent Literature 2: JP 2014-155693 A

### SUMMARY OF INVENTION

### Technical Problem

Conventionally, there has been a problem that to perform gait analysis or to detect a motion of a person, high-cost special equipment such as a depth camera and a marker attached to a person is required. Further, conventionally, as a condition for fatigue determination, only feature information such as a right-left ratio of a stride and an arm swing angle is listed. It is not indicated what kind of change is effective for fatigue determination. This poses a problem that a detection effectiveness is low.

An objective of the present invention is to provide a fatigue determination device that can be introduced at a low cost and with ease, and can accurately determine fatigue.

### Solution to Problem

A fatigue determination device according to the present invention includes:

a skeleton extraction unit to extract skeleton information expressing in a time series a motion of a skeleton of a person from two-dimensional video data obtained by capturing a walking movement of the person;

a walk analysis unit to calculate walk analysis data including arm swing information expressing an arm swing state of the person in walking and gait information expressing a gait state of the person in walking, using the skeleton information; and

a determination unit to compare a determination threshold value for determining a fatigue degree of the person with the walk analysis data of the person, and to determine the fatigue degree of the person using a comparison result, the determination threshold value including a threshold value of the arm swing information and a threshold value of the gait information.

### Advantageous Effects of Invention

In a fatigue determination method according to the present invention, a skeleton extraction unit extracts skeleton information expressing in a time series a motion of a skeleton of a person from two-dimensional video data obtained by capturing a walking movement of the person. A walk analysis unit calculates walk analysis data including arm swing information expressing an arm swing state of the person in walking and gait information expressing a gait state of the person in walking, using the skeleton information. Then, a determination unit compares a determination threshold value including a threshold value of the arm swing information and a threshold value of the gait information, with the walk analysis data of the person, and determines a fatigue degree of the person using a comparison result. Hence, with the fatigue determination device according to the present invention, it is possible to realize a fatigue determination device that can be introduced at a low cost and with ease, and can accurately determine fatigue.

### BRIEF DESCRIPTION OF DRAWINGS

FIG. 1 presents an application example of a fatigue determination device according to Embodiment 1.

FIG. 2 is a configuration diagram of the fatigue determination device according to Embodiment 1.

FIG. 3 is a flowchart illustrating operations of the fatigue determination device according to Embodiment 1.

FIG. 4 presents diagrams illustrating traces of time-series skeleton information according to Embodiment 1.

FIG. 5 is a diagram illustrating an example of walk analysis processing according to Embodiment 1.

FIG. 6 is a diagram illustrating another example of the walk analysis processing according to Embodiment 1.

FIG. 7 presents examples of calculating a foot fluctuation and calculating a change amount in foot separation width, width with respect to a traveling direction according to Embodiment 1.

### DESCRIPTION OF EMBODIMENTS

An embodiment of the present invention will now be described with referring to drawings. In the drawings, the same or equivalent portion is denoted by the same reference sign. In description of the embodiment, the same or equivalent portion will not be described or will be described briefly, as needed. Description of the embodiment may sometimes indicate an orientation or position such as above, below, left, right, forth, back, front, and rear. These notations are employed for the sake of descriptive convenience and do not limit a layout, a direction, or orientation of a device, a tool, a component, or the like.

### Embodiment 1

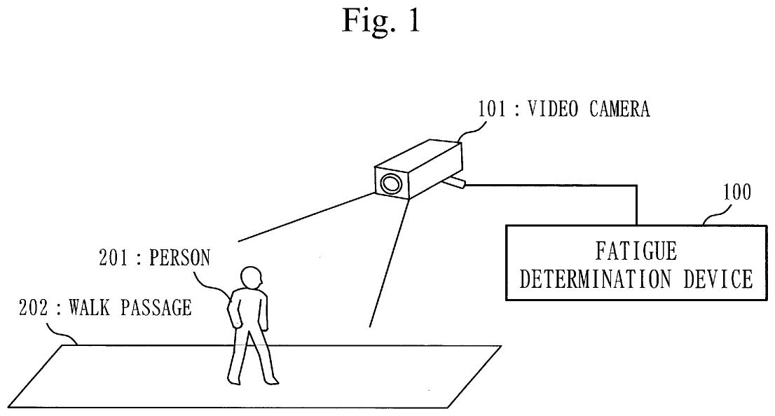

FIG. 1 is a diagram illustrating an application example of a fatigue determination device 100 according to the present embodiment.

FIG. 1 presents an example in which the fatigue determination device 100 according to the present embodiment is installed midway along a walk passage 202 of a person 201.

A video camera 101 is set at a position where it can photograph the person 201 walking on the walk passage 202. The video camera 101 acquires a video of walking of the person 201 when the person 201 is walking on the walk passage 202. The video of walking acquired by the video camera 101 is inputted to the fatigue determination device 100.

The fatigue determination device 100 performs fatigue determination of the person 201 using the video of walking. A determination result is notified to a portable terminal device such as a smartphone or tablet owned by the person 201. Alternatively, the determination result may be notified to an organization such as health insurance association of an institution the person 201 belongs to. In this manner, a fatigue status of the person 201 determined by the fatigue determination device 100 can be utilized widely.

The person 201 need not be aware that the video camera 101 is installed. This signifies that there is no restriction at all such as requesting cooperation from the person 201. Namely, fatigue determination can be practiced anytime in a daily life wherever a camera is installed. Furthermore, the video camera 101 to be used for video acquisition need not be a special camera such as a depth camera, but a camera such as a surveillance camera already existing in the society can be utilized.

The video camera 101 can be arranged at any position as far as it can photograph the person 201. The video camera 101 and the fatigue determination device 100 may be connected to each other by wired connection or wireless connection. If real-time information communication is not required, a video acquired by the video camera 101 may be accumulated in a recording medium, or may be inputted to the fatigue determination device 100 off-line. Therefore, the fatigue determination device 100 may be installed at a location remote from the video camera 101.

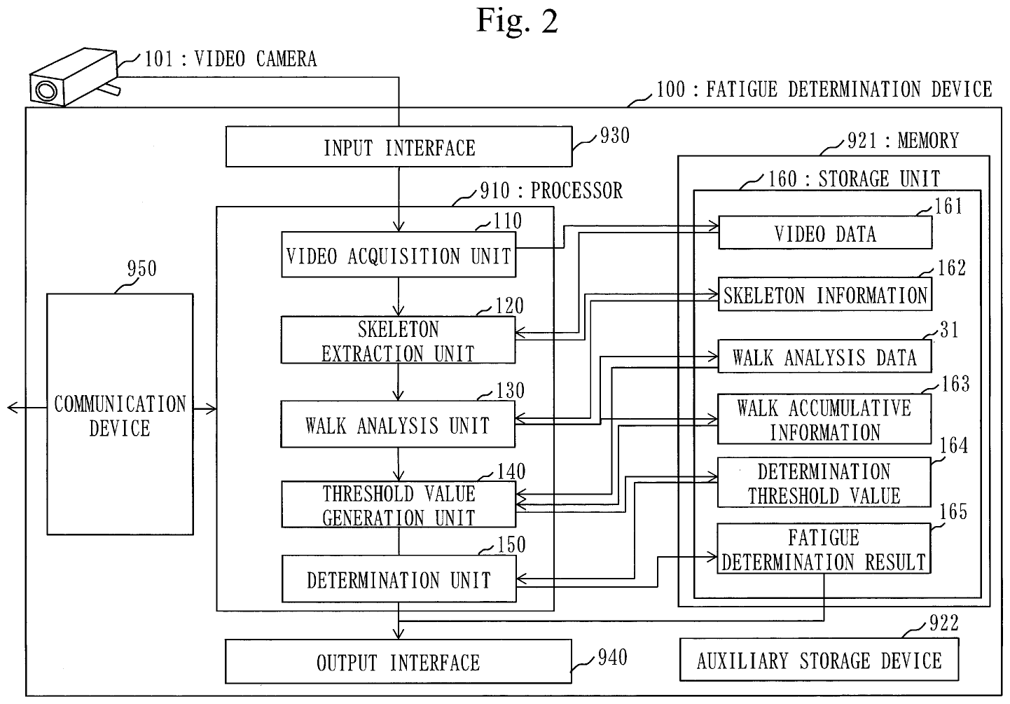

A configuration of the fatigue determination device 100 according to the present embodiment will be described with referring to FIG. 2.

The fatigue determination device 100 is a computer. The fatigue determination device 100 is provided with a processor 910, and is provided with other hardware devices such as a memory 921, an auxiliary storage device 922, an input interface 930, an output interface 940, and a communication device 950. The processor 910 is connected to the other hardware devices via a signal line and controls the other hardware devices.

The fatigue determination device 100 is provided with a video acquisition unit 110, a skeleton extraction unit 120, a walk analysis unit 130, a threshold value generation unit 140, a determination unit 150, and a storage unit 160, as function elements. Video data 161, skeleton information 162, walk analysis data 31, walk accumulative information 163, a determination threshold value 164, and a fatigue determination result 165 are stored in the storage unit 160.

Functions of the video acquisition unit 110, skeleton extraction unit 120, walk analysis unit 130, threshold value generation unit 140, and determination unit 150 are implemented by software. The storage unit 160 is provided to the memory 921.

The processor 910 is a device that executes a fatigue determination program. The fatigue determination program is a program that implements the functions of the video acquisition unit 110, skeleton extraction unit 120, walk analysis unit 130, threshold value generation unit 140, and determination unit 150.

The processor 910 is an Integrated Circuit (IC) that performs computation processing. Specific examples of the processor 910 include a CPU, a Digital Signal Processor (DSP), and a Graphics Processing Unit (GPU).

The memory 921 is a storage device that stores data temporarily. Specific examples of the memory 921 include a Static Random-Access Memory (SRAM) and a Dynamic Random-Access Memory (DRAM).

The auxiliary storage device 922 is a storage device that keeps data. Specific examples of the auxiliary storage device 922 include an HDD. Alternatively, the auxiliary storage device 922 may be a portable storage medium such as an SD (registered trademark) memory card, a CF, a NAND flash, a flexible disk, an optical disk, a compact disk, a Blu-ray (registered trademark) Disc, and a DVD. Note that HDD stands for Hard Disk Drive; SD (registered trademark) stands for Secure Digital; CF stands for CompactFlash (registered trademark); and DVD stands for Digital Versatile Disk.

The input interface 930 is a port to be connected to an input device such as a mouse, a keyboard, and a touch panel. The input interface 930 is specifically a Universal Serial Bus (USB) terminal. The input interface 930 may be a port to be connected to a Local Area Network (LAN). The fatigue determination device 100 is connected to the video camera 101 via the input interface 930.

The output interface 940 is a port to which a cable of an output apparatus such as a display is connected. The output interface 940 is specifically a USB terminal or a High-Definition Multimedia Interface (HDMI; registered trademark) terminal. The display is specifically a Liquid Crystal Display (LCD).

The communication device 950 has a receiver and a transmitter. The communication device 950 is connected to a communication network such as a LAN, the Internet, and a telephone line by wireless connection. The communication device 950 is specifically a communication chip or a Network Interface Card (NIC).

The fatigue determination program is read into the processor 910 and executed by the processor 910. Not only the fatigue determination program but also an Operating System (OS) is stored in the memory 921. The processor 910 executes the fatigue determination program while executing the OS. The fatigue determination program and the OS may be stored in the auxiliary storage device 922. The fatigue determination program and the OS stored in the auxiliary storage device 922 are loaded to the memory 921 and executed by the processor 910. The fatigue determination program may be incorporated in the OS partly or entirely.

The fatigue determination device 100 may be provided with a plurality of processors that substitute for the processor 910. The plurality of processors share execution of the fatigue determination program. Each processor is a device that executes the fatigue determination program just as the processor 910 does.

Data, information, signal values, and variable values utilized, processed, or outputted by the fatigue determination program are stored in the memory 921, the auxiliary storage device 922, or in a register or cache memory in the processor 910.

A word “unit” in each of the video acquisition unit 110, the skeleton extraction unit 120, the walk analysis unit 130, the threshold value generation unit 140, and the determination unit 150 may be replaced by “process”, “procedure”, or “stage”. A word “process” in each of a video acquisition process, a skeleton extraction process, a walk analysis process, a threshold value generation process, and a determination process may be replaced by “program”, “program product”, or “computer-readable recording medium recorded with a program”.

The fatigue determination program causes the computer to execute each process, each procedure, or each stage corresponding to the above individual unit with its “unit” being replaced by “process”, “procedure”, or “stage”. The fatigue determination method is a method carried out as the fatigue determination device 100 executes the fatigue determination program.

The fatigue determination program may be presented as being stored in a computer-readable recording medium. The fatigue determination program may be presented as a program product.

The hardware configuration of the fatigue determination device 100 of FIG. 2 is presented as an example and may be subject to addition, deletion, or exchange according to an embodiment. For example, if the video camera 101 is built in the fatigue determination device 100, the input interface 930 may be unnecessary. For example, if the fatigue determination device 100 incorporates a display that displays the fatigue determination result 165, the output interface 940 may be unnecessary. For example, the auxiliary storage device 922 storing information such as the fatigue determination program and the walk accumulative information 163 may exist outside the fatigue determination device 100 and may be connected to the fatigue determination device 100 via an input/output interface. For example, the fatigue determination device 100 may have an input interface with a plurality of inputs for connecting a plurality of video cameras to the fatigue determination device 100.

***Description of Operations***

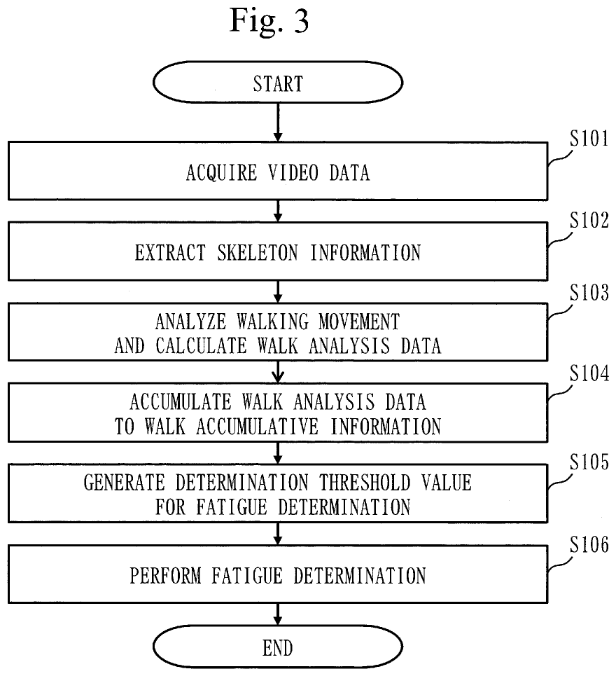

Operations of the fatigue determination device 100 according to the present embodiment will be described with referring to FIG. 3.

<Image Acquisition Process>

In step S101, the video acquisition unit 110 acquires, via the input interface 930, the video data 161 captured by the video camera 101. The video camera 101 is installed at a position to photograph the person 201. The video data 161 is two-dimensional video data obtained by capturing a walking movement of the person 201. The video camera 101 may be specifically a camera such as a surveillance camera widely installed in the community. The video data 161 is specifically a two-dimensional color video. The video data 161 is outputted to the skeleton extraction unit 120.

<Skeleton Extraction Process>

In step S102, the skeleton extraction unit 120 extracts the skeleton information 162 expressing in a time series a motion of a skeleton of the person 201 from the two-dimensional video data 161 obtained by capturing the walking movement of the person 201. The skeleton extraction unit 120 extracts the three-dimensional skeleton information 162 from the video data 161. Because of development of advanced computer vision technology in recent years, the skeleton information 162 can be extracted from two-dimensional video data having no depth information. The skeleton extraction unit 120 extracts the person 201 appearing in the video data 161 and extracts the time-series skeleton information 162 of the extracted person using the advanced computer vision technology.

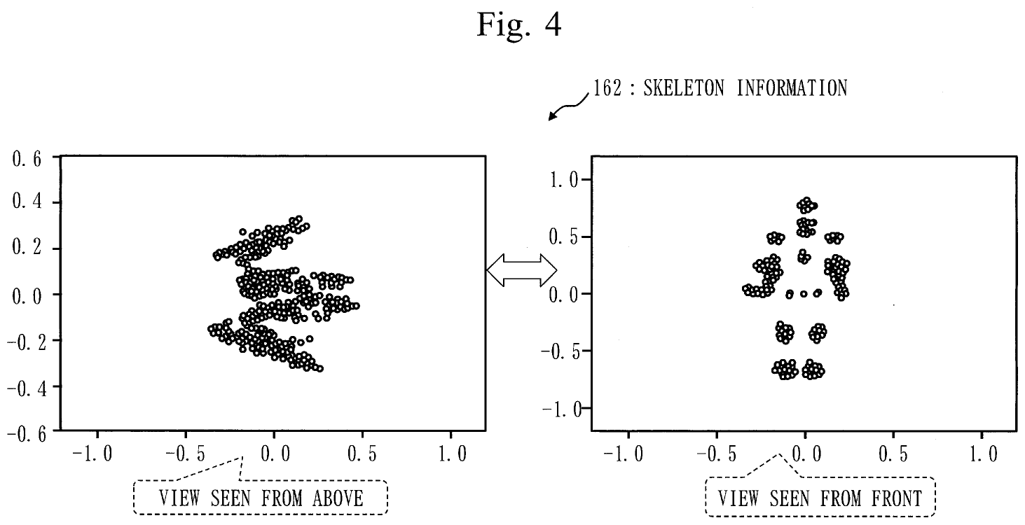

FIG. 4 presents diagrams illustrating traces of the time-series skeleton information 162 according to the present embodiment.

Specifically, the skeleton extraction unit 120 extracts the skeleton information 162 using a technique such as OpenPose and DepthPose. The technique such as OpenPose and DepthPose is a deep-learning algorithm that extracts skeleton information from a video. Using such deep-learning algorithm and its model, the skeleton extraction unit 120 executes processing on a video of the person contained in the video data 161, and obtains the skeleton information 162 as a processing result. In the present circumstances, OpenPose and DepthPose are known well each as the algorithm to extract the skeleton information. However, the skeleton extraction unit 120 can also introduce a new skeleton extraction algorithm to be developed in the future.

The skeleton information 162 is not necessarily three-dimensional information but may be two-dimensional information obtained by projecting three-dimensional information onto a plane. The skeleton information 162 is outputted to the walk analysis unit 130.

<Walk Analysis Process>

An outline of operations of the walk analysis unit 130 will be described.

The walk analysis unit 130 calculates the walk analysis data 31 including arm swing information 611 and gait information 612, using the skeleton information 162. The arm swing information 611 expresses an arm swing state of the person 201 in walking. The gait information 612 expresses a gait state of the person 201 in walking.

The walk analysis unit 130 calculates, as the arm swing information 611, an arm swing angle of the person 201 with respect to a traveling direction, and an arm swing magnitude of the person. The arm swing angle of the person 201 with respect to the traveling direction may be expressed as an arm swing angle in a right-and-left direction. The arm swing magnitude of the person 201 may be expressed as an arm swing angle in a back-and-forth direction.

The walk analysis unit 130 also calculates, as the gait information 612, a magnitude of foot fluctuation of the person and a change amount in foot separation width of the person, with respect to the traveling direction.

In step S103, the walk analysis unit 130 analyzes the walking movement of the person 201 on the basis of the skeleton information 162. The walk analysis unit 130 outputs an analysis result as the walk analysis data 31. The walk analysis data 31 includes specifically information such as the skeleton information, the arm swing information 611, and the gait information 612 which are subject to position correction with using hip position information.

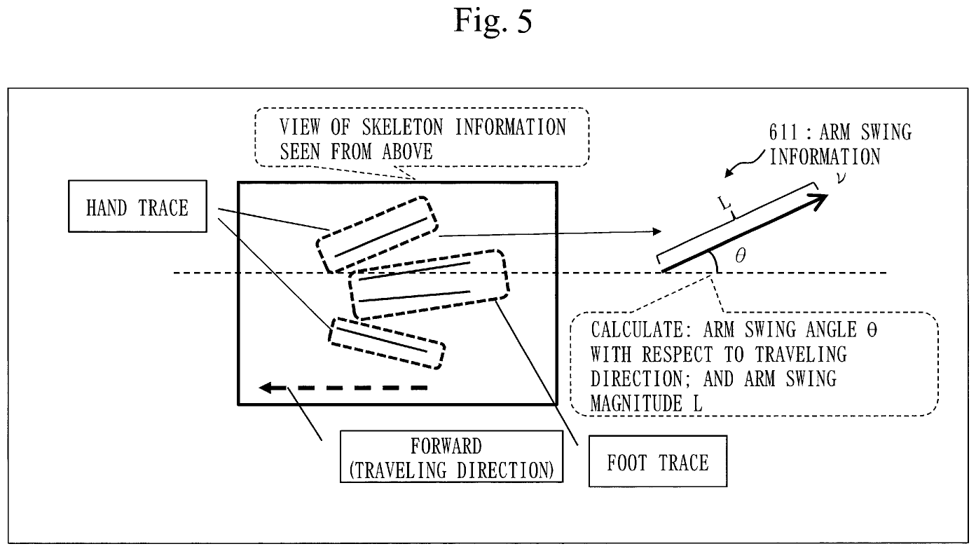

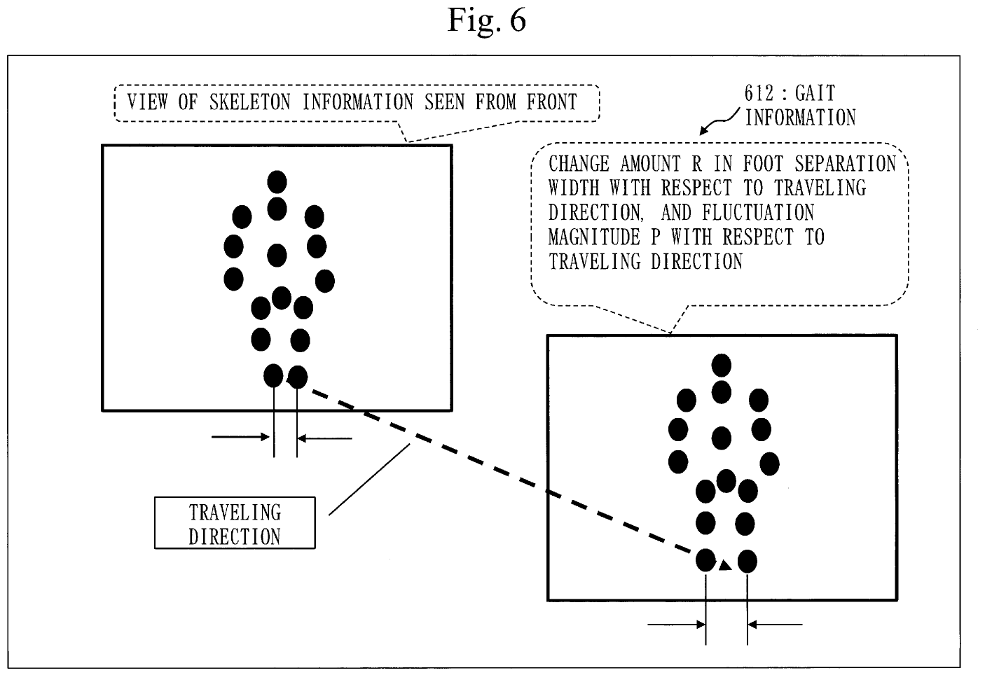

FIG. 5 and FIG. 6 are diagrams illustrating examples of processing by the walk analysis unit 130 according to the present embodiment.

The walk analysis unit 130 takes as input the time-series skeleton information 162 and analyzes an angle or magnitude of arm swing in the back-and-forth direction and an angle or magnitude of arm swing in the right-and-left direction. The walk analysis unit 130 also takes as input the time-series skeleton information 162 and analyzes the walking movement such as left-and-right fluctuation of gait and a change in foot separation width, with respect to the traveling direction.

FIG. 5 is a schematic diagram, seen from above the head, of 3 walk cycles of the skeleton information 162. Information of the hand trace and information of the foot trace of FIG. 5 can be expressed as angle information and length information with respect to the traveling direction. When fatigue occurs, walk of the person becomes unstable. As compared with walk without fatigue, the arm swing becomes large on both sides in order to compensate for unstable walk. Also, it is observed that the person tends to swing his arms largely. In view of this, the arm swing information 611 including an arm swing angle θ with respect to the traveling direction and an arm swing magnitude L serves as information to express the fatigue of the person more directly. The arm swing magnitude L may be expressed as an arm swing angle in the back-and-forth direction. The arm swing angle θ with respect to the traveling direction may be expressed as an arm swing angle in the right-and-left direction.

FIG. 6 is a schematic diagram, seen from the traveling direction, of the 3 walk cycles of the skeleton information 162. The information of the foot trace in FIGS. 5 and 6 can be expressed as information of a magnitude of fluctuation in foot position and as information of spread of the both feet when walking in the traveling direction. When fatigue occurs, it destabilizes the walk of the person and makes it difficult for the person to walk straight in the traveling direction. It is then observed that the person tends to secure stability by walking zigzag, or by walking with a wide stride for securing stability. Therefore, the gait information 612, including a foot fluctuation width P and a change amount R in foot separation width, with respect to the traveling direction when walking in the traveling direction, serves as information that expresses the fatigue of the person more directly.

The walk analysis unit 130 utilizes characteristics of the walking movement in fatigue described above, and calculates the walk analysis data 31 as the information that expresses the fatigue more directly.

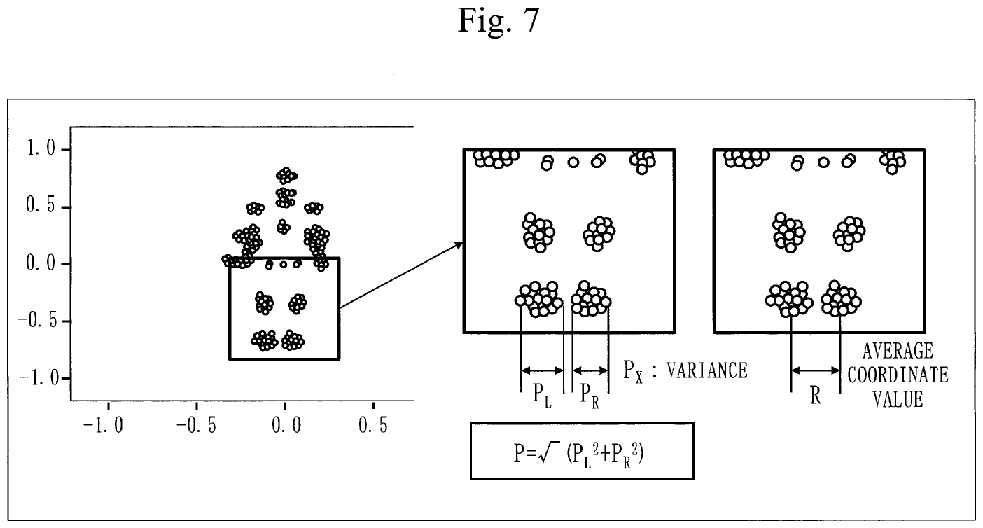

Example of calculating the foot fluctuation width P and calculating the change amount R in foot separation width, with respect to the traveling direction will be described with referring to FIG. 7.

With referring to FIG. 7, description will be made on an example of calculating the foot fluctuation width P and the change amount R in foot separation width, with respect to the traveling direction with using information of a foot portion of FIG. 4 which is seen from the front.

The foot fluctuation width P with respect to the traveling direction may be obtained as an L2 norm, as P=√(P_{L}²+P_{R}²) where P_{L} is variance (P_{X}) of a fluctuation width of a foot on the left side in the drawing, and P_{R} is variance (P_{X}) of a fluctuation width of a foot on the right side in the drawing.

Alternatively, the change amount R in foot separation width may be obtained by defining R as a change amount in average value of coordinates of each of the both feet.

Note that the calculating expression of P_{X} variance and P (L2 norm) is presented as an example. Alternatively, P_{X} may be calculated by another method in which, for example, P_{X} is a difference between the maximum and the minimum, or is an event probability. The calculating expression of P may be L1 norm (sum of absolute values).

This also applies to the average coordinate value to be used for calculating the change amount R. A median may be used as the average coordinate value to be used for calculation of the change amount R.

In this manner, the foot fluctuation width P and the change amount R in foot separation width, with respect to the traveling direction may be calculated in any calculation method as far as P and R can be expressed appropriately.

As described above, specifically, the walk analysis unit 130 calculates information of the angle θ and magnitude L of arm swing, and information of the change amount R in foot separation width and the magnitude P of fluctuation, with respect to the traveling direction, as the walk analysis data 31 being analytical information of the walking movement. The information of the angle and magnitude of arm swing, and the information of the change amount in foot separation width and the magnitude of fluctuation, with respect to the traveling direction include information such as a magnitude and angle of the arm swing in the back-and-forth direction and right-and-left direction, and rightward-and-leftward fluctuation of the gait and a foot separation width of the gait, with respect to the traveling direction.

In FIGS. 5 and 6, the information of the angle and magnitude of arm swing and the information of the change amount in foot separation width and the magnitude of fluctuation, with respect to the traveling direction are treated as the walk analysis data 31. However, these pieces of information can be expressed in a different manner. For example, the information can be expressed by a two-dimensional vector in place of a length and an angle. For example, the information of the fluctuation can be expressed as standard deviation or variance.

In step S104, the walk analysis unit 130 stores the walk analysis data 31 to the storage unit 160 and accumulates the walk analysis data 31 to the walk accumulative information 163.

<Threshold Value Generation Process>

In step S105, the threshold value generation unit 140 generates the determination threshold value 164 to be used for fatigue determination. The threshold value generation unit 140 generates the determination threshold value 164 including a threshold value of the arm swing information 611 and a threshold value of the gait information 612, using the walk accumulative information 163 in which walk analysis data calculated formerly by the walk analysis unit 130 is accumulated. The threshold value generation unit 140 generates the determination threshold value 164 by combining the walk analysis data accumulated formerly and the walk analysis data 31 calculated this time. The walk analysis data accumulated formerly and the walk analysis data 31 calculated this time do not necessarily belong to the same person. Meanwhile, if it is known in advance that the former walk analysis data and the walk analysis data 31 of this time belong to the same person, a determination threshold value 164 having a higher accuracy can be generated. In this manner, the threshold value generation unit 140 can also correlate the walk analysis data to be inputted, with the person. This correlation can be realized by a method of performing correlation with an individual at the time of capturing with the video camera 101, or by a method of identifying an individual in the video acquisition unit 110 using biometrics such as a face and a gait.

The threshold value generation unit 140 generates the determination threshold value 164 by carrying out clustering on the basis of whether fatigue exists or not, using the walk analysis data calculated so far. The determination threshold value 164 includes, for example, a threshold value of the information of the angle and magnitude of arm swing, and a threshold value of the information of a change in foot separation width and the magnitude of fluctuation, with respect to the traveling direction. That is, the determination threshold value 164 includes the threshold value of the arm swing information 611 and the threshold value of the gait information 612. The threshold value generation unit 140 generates the determination threshold value 164 each time the walk analysis unit 130 calculates the walk analysis data 31.

When a number of pieces of walk analysis data exist that are sufficient for a clustering process for generating the determination threshold value 164, it is possible to omit the process of generating the determination threshold value 164. The threshold value generation unit 140 may generate the determination threshold value 164 periodically or non-periodically, and may store the determination threshold value 164 in the storage unit 160. Then, the determination unit 150 may perform the determination process using the determination threshold value 164 stored in the storage unit 160.

<Determination Process>

In step S106, the determination unit 150 compares the determination threshold value 164 with the walk analysis data 31 of the person 201, and determines a fatigue degree of the person 201 using a comparison result. The determination threshold value 164 is used for determining the fatigue degree of the person. The determination threshold value 164 includes the threshold value of the arm swing information 611 and the threshold value of the gait information 612. Specifically, the determination unit 150 compares the information of the angel and magnitude of arm swing and the information of the change in foot separation width and of the magnitude of fluctuation, with respect to the traveling direction, which are included in the walk analysis data 31 of the person 201, with the determination threshold value 164. The determination unit 150 determines the fatigue degree of the person 201 from a comparison result. The determination unit 150 outputs a determination result as the fatigue determination result 165, to the output apparatus such as the display via the output interface 940.

Assume a case wherein, of the walk analysis data 31, each of the arm swing angle θ with respect to the traveling direction, the arm swing magnitude L, the gait fluctuation width P, and the change amount R in foot separation width is compared with a corresponding determination threshold value 164. If every data is less than the corresponding determination threshold value 164, it is determined that the fatigue degree of the person 201 is 0 to 2. If there is one or two pieces of data each being equal to or more than the corresponding determination threshold value 164, it is determined that the fatigue degree of the person 201 is 3 to 5. If there are three pieces of data each being equal to or more than the corresponding determination threshold value 164, it is determined that the fatigue degree of the person 201 is 6 to 8. If there are four pieces of data each being equal to or more than the corresponding determination threshold value 164, that is, if every data is equal to or more than the corresponding determination threshold value 164, it is determined that the fatigue degree of the person 201 is 9 to 10. Alternatively, weighting may be performed in units of data. For example, if the fluctuation in the gait is large, the person 201 is supposed to be much more tired. Thus, the fatigue degree may be determined after the gait fluctuation width P is weighted.

In the above description, separate determination threshold values are prepared for the arm swing angle, the arm swing width, the gait fluctuation magnitude, and the change amount in foot separation width individually. Alternatively, for example, the information of the arm swing angle, the information of the arm swing magnitude, the information of the gait fluctuation width, and the information of the change amount in foot separation width may be integrated after they are weighted by the individual weights, and an integration result may be subjected to determination using one or a plurality of threshold values. Such a determination method is employed in a neural network.

The determination unit 150 determines the fatigue degree of the person 201. Alternatively, the determination unit 150 may merely determine whether or not the person 201 is tired.

The determination unit 150 may compare the arm swing angle in the back-and-forth direction and the arm swing angle in the right-and-left direction, and then determine whether or not the person 201 is tired from a comparison result. For example, it may be determined that the person 201 is tired when the arm swing angle in the right-and-left direction becomes larger than the arm swing angle in the back-and-forth direction.

A processing procedure concerning fatigue determination described above is presented as an example. Each process may be subject to omission, exchange, or addition of a processing procedure as far as the fatigue determination result 165 to be outputted can be obtained.

***Other Configurations***

### <Modification 1>

In the present embodiment, the functions of the video acquisition unit 110, skeleton extraction unit 120, walk analysis unit 130, threshold value generation unit 140, and determination unit 150 are implemented by software. According to a modification, the functions of the video acquisition unit 110, skeleton extraction unit 120, walk analysis unit 130, threshold value generation unit 140, and determination unit 150 may be implemented by hardware.

The fatigue determination device 100 is provided with an electronic circuit in place of the processor 910.

The electronic circuit is a dedicated electronic circuit that implements the functions of the video acquisition unit 110, skeleton extraction unit 120, walk analysis unit 130, threshold value generation unit 140, and determination unit 150.

The electronic circuit is specifically a single circuit, a composite circuit, a programmed processor, a parallel-programmed processor, a logic IC, a GA, an ASIC, or an FPGA. Note that GA stands for Gate Array; ASIC stands for Application Specific Integrated Circuit; and FPGA stands for Field-Programmable Gate Array.

The functions of the video acquisition unit 110, skeleton extraction unit 120, walk analysis unit 130, threshold value generation unit 140, and determination unit 150 may be implemented by one electronic circuit, or by a plurality of electronic circuit through distribution among them.

According to another modification, the functions of some of the video acquisition unit 110, skeleton extraction unit 120, walk analysis unit 130, threshold value generation unit 140, and determination unit 150 may be implemented by an electronic circuit, and the functions of the remaining units may be implemented by software.

The processor and the electronic circuit are called processing circuitry as well. That is, in the fatigue determination device 100, the functions of the video acquisition unit 110, skeleton extraction unit 120, walk analysis unit 130, threshold value generation unit 140, and determination unit 150 are implemented by processing circuitry.

***Description of Effect of Present Embodiment***

In the fatigue determination device 100, the walking movement is analyzed with using two-dimensional video data. Therefore, fatigue determination can be practiced anytime in a daily life wherever a camera is installed. The camera need not be a special camera such as a depth camera, but a surveillance camera already existing in the society can be utilized. Hence, with the fatigue determination device 100 according to the present embodiment, a fatigue determination device that can be introduced at a low cost and with ease can be realized.

With the fatigue determination device 100 according to the present embodiment, a determination threshold value is generated each time walk analysis data is analyzed with using the walk accumulative information in which former walk analysis data is accumulated. Hence, with the fatigue determination device 100 according to the present embodiment, more accurate, highly precise fatigue determination can be performed.

In the fatigue determination device 100 according to the present embodiment, the skeleton extraction unit extracts three-dimensional time-series skeleton information from two-dimensional video data. The walk analysis unit gets a grasp of body movement more accurately using the three-dimensional time-series skeleton information. Therefore, with the fatigue determination device 100 according to the present embodiment, more accurate, highly precise fatigue determination can be performed.

In above Embodiment 1, each unit of the fatigue determination device is described as an independent function block. However, the configuration of the fatigue determination device is not necessarily a configuration as that in the embodiment described above. A function block of the fatigue determination device can be of any configuration as far as it can implement the function described in the embodiment described above. The fatigue determination device is not necessarily one device but may be a system formed of a plurality of devices.

A plurality of portions of Embodiment 1 may be practiced by combination. One portion of the present embodiment may be practiced. Also, the present embodiment may be practiced entirely or partly by any combination.

That is, in Embodiment 1, some portions of the embodiment can be combined arbitrarily, an arbitrary constituent element of the embodiment can be modified, or an arbitrary constituent element of the embodiment can be omitted.

The embodiment described above is an essentially preferable exemplification and is not intended to limit the scope of the present invention, the scope of an applied product of the present invention, and the scope of usage of the present invention. Various changes can be made in the embodiment described above as necessary.

### REFERENCE SIGNS LIST

31: walk analysis data; 100: fatigue determination device; 101: video camera; 110: video acquisition unit; 120: skeleton extraction unit; 130: walk analysis unit; 140: threshold value generation unit; 150: determination unit; 160: storage unit; 161: video data; 162: skeleton information; 163: walk accumulative information; 164: determination threshold value; 165: fatigue determination result; 201: person; 202: walk passage; 611: arm swing information; 612: gait information; 910: processor; 921: memory; 922: auxiliary storage device; 930: input interface; 940: output interface; 950: communication device.

1. A fatigue determination device comprising:
processing circuitry
to extract skeleton information expressing in a time series a motion of a skeleton of a person from two-dimensional video data obtained by capturing a walking movement of the person,
to calculate walk analysis data including arm swing information expressing an arm swing state of the person in walking and gait information expressing a gait state of the person in walking, using the skeleton information, and
to compare a determination threshold value for determining a fatigue degree of the person with the walk analysis data of the person, and to determine the fatigue degree of the person using a comparison result, the determination threshold value including a threshold value of the arm swing information and a threshold value of the gait information,
wherein the processing circuitry calculates, as the arm swing information, an arm swing angle of the person with respect to a traveling direction, and an arm swing magnitude of the person.

2. A fatigue determination device comprising:
processing circuitry
to extract skeleton information expressing in a time series a motion of a skeleton of a person from two-dimensional video data obtained by capturing a walking movement of the person,
to calculate walk analysis data including arm swing information expressing an arm swing state of the person in walking and gait information expressing a gait state of the person in walking, using the skeleton information, and
to compare a determination threshold value for determining a fatigue degree of the person with the walk analysis data of the person, and to determine the fatigue degree of the person using a comparison result, the determination threshold value including a threshold value of the arm swing information and a threshold value of the gait information,
wherein the processing circuitry calculates, as the gait information, a magnitude of foot fluctuation of the person and a change amount in foot separation width of the person, with respect to a traveling direction.

3. The fatigue determination device according to claim 2,
wherein the processing circuitry calculates, as the arm swing information, an arm swing angle of the person with respect to a traveling direction, and an arm swing magnitude of the person.

4. The fatigue determination device according to claim 1,
wherein the processing circuitry generates the determination threshold value including the threshold value of the arm swing information and the threshold value of the gait information, using walk accumulative information in which walk analysis data calculated formerly is accumulated.

5. The fatigue determination device according to claim 1,
wherein the processing circuitry generates the determination threshold value each time the walk analysis data is calculated.

6. The fatigue determination device according to claim 1,
wherein the processing circuitry extracts three-dimensional skeleton information from the video data.

7. A fatigue determination method of a fatigue determination device, the fatigue determination method comprising:
extracting skeleton information expressing in a time series a motion of a skeleton of a person from two-dimensional video data obtained by capturing a walking movement of the person;
calculating walk analysis data including arm swing information expressing an arm swing state of the person in walking and gait information expressing a gait state of the person in walking, using the skeleton information; and
comparing a determination threshold value for determining a fatigue degree of the person with the walk analysis data of the person, and determining the fatigue degree of the person using a comparison result, the determination threshold value including a threshold value of the arm swing information and a threshold value of the gait information,
wherein the calculating walk analysis data includes calculating, as the arm swing information, an arm swing angle of the person with respect to a traveling direction, and an arm swing magnitude of the person.

8. A fatigue determination method of a fatigue determination device, the fatigue determination method comprising:
extracting skeleton information expressing in a time series a motion of a skeleton of a person from two-dimensional video data obtained by capturing a walking movement of the person;
calculating walk analysis data including arm swing information expressing an arm swing state of the person in walking and gait information expressing a gait state of the person in walking, using the skeleton information; and
comparing a determination threshold value for determining a fatigue degree of the person with the walk analysis data of the person, and determining the fatigue degree of the person using a comparison result, the determination threshold value including a threshold value of the arm swing information and a threshold value of the gait information,
wherein the calculating walk analysis data includes calculating, as the gait information, a magnitude of foot fluctuation of the person and a change amount in foot separation width of the person, with respect to a traveling direction.
